(19) United States
(12) Patent Application Publication
(10) Pub. No.: US 2010/0266577 A1
(43) Pub. Date: Oct. 21, 2010

(54) Method of Cancer Treatment with Antagonists of FAS Inhibitors

(76) Inventors: Felipe Samaniego, Houston, TX (US); Suizhao Wang, Pearland, TX (US); Shu Wang, Houston, TX (US)

Correspondence Address: WILLIAMS, MORGAN & AMERSON, 10333 RICHMOND, SUITE 1100, HOUSTON, TX, 77042, US

(21) Appl. No.: 12/670,224

(22) PCT Filed: Jul. 23, 2008
(86) PCT No.: PCT/US08/08914

§ 371 Date: Jul. 1, 2010

Related U.S. Application Data
(60) Provisional application No. 60/951,763, filed on Jul. 25, 2007.

(51) Int. Cl. A61K 39/395; A61K 38/00; C07K 14/00; C07K 16/00; C07H 21/04; A61P 35/00

(52) U.S. Cl. 424/130.1; 514/19.3; 530/350; 530/387.1; 536/23.1

(57) ABSTRACT

In one embodiment, the present invention relates to a method of treating a cancer characterized by inhibition of Fas-mediated apoptosis, comprising administering to a patient suffering from the cancer a pharmaceutically-effective amount of a composition comprising a peptide that competitively antagonizes at least one protein-Fas binding selected from the group consisting of herpes virus 8 protein K1 binding to Fas, CD74 binding to Fas, and pi 00 binding to Fas. In a further embodiment, the peptide is selected from the group consisting of peptides having from about 15 to about 35 amino acid residues and comprising at least a portion of the immunoglobulin domain of K1, peptides having from about 15 to about 35 amino acid residues and comprising at least fifteen consecutive amino acid residues among positions 50-120 of CD74, LVTLLLAGQ ATT A YFL YQQQ, LYQQQGRLDKLTVTSQNLQL, QNLQLENLRMKLPKPPKPVS, and PKPVSKMRMATPLLMQALPM. In another embodiment, the present invention relates to a method of treating a disease characterized by unwanted apoptosis in a mammal, comprising transfecting a cell of the mammal with a nucleic acid molecule encoding CD74. The present invention also relates to the compositions referred to above and their use in manufacture of medicaments for treatment of the diseases described herein.

### BACKGROUND OF THE INVENTION

The present invention relates generally to the field of cancer treatment. More particularly, it concerns chemotherapeutic treatments for cancers characterized by inhibition of Fas-mediated apoptosis.

### SUMMARY OF THE INVENTION

In one embodiment, the present invention relates to a method of treating a cancer characterized by inhibition of Fas-mediated apoptosis, comprising administering to a patient suffering from the cancer a pharmaceutically-effective amount of a composition comprising a peptide that competitively antagonizes at least one protein-Fas binding selected from the group consisting of herpes virus 8 protein K1 binding to Fas, CD74 binding to Fas, and p100 binding to Fas.

In another embodiment, the present invention relates to a method of treating a disease characterized by unwanted apoptosis in a mammal, comprising transfecting a cell of the mammal with a nucleic acid molecule encoding CD74.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Human Herpesvirus 8 K1 and cellular proteins p100 and CD74 are candidate inhibitors of the death receptor Fas. K1, p100 and CD74 interact with Fas to suppress the Fas-mediated (extrinsic) pathway of apoptosis. The second pathway is death mediated through the mitochondria (intrinsic). Chemotherapy induced cancer regression uses these to pathways to kill cells.

FIG. 2. K1 is associated with suppressed procaspase 8 activation with Fas. A) K1-expressing cells had lower levels of procaspase 8 cleavage than BJABK1m and BJABXS cells incubated with apoptosis-inducing anti-Fas antibody. B) BJAB cells were transiently transfected with plasmids expressing K1myc and Fas, as shown across the tops of the lanes. Immunoprecipitation/immunoblotting analyses were performed with anti-Fas or anti-myc antibodies.

FIG. 3. K1-transfected mouse liver was protected from Fas-induced apoptosis. Mice were transfected with plasmid expressing K1Flag. The livers of K1-transfected mice showed less hemorrhaging than vector control-transfected mice induced to undergo apoptosis by inoculation with agonistic anti-Fas antibody. TUNEL staining showed K1 protecting Fas-mediated apoptosis in transfected mouse tissues.

FIG. 4. The immunoglobulin domain of K1 is used to bind to Fas and peptides representing the binding domain overexpression of an Ig containing protein (CD79b) competes K1-Fas complex. (A) Cells were transfected with plasmid expressing CD79b, HA-Fas or FlagK1. (B) Protein associations were demonstrated in immunoprecipitation/immunoblotting analysis. Over expression of K1 overcame the effects of CD79a expression. Plasmid vector DNA was adjusted to keep the total DNA constant.

FIG. 5. K1 sequesters Fas and prevents participation in DISC formation. Cells were activated with CH-11 antibody and the extracts were immunodepleted with the CH-11 antibody. The left over supernatant was precipitated a second time with anti-Fas antibody (B-10) that binds to all Fas (inactivated Fas and intracellularly located Fas at time of first CH-11 antibody incubation. We found that in K1 cells have fewer Fas at the DISC and most of Fas in away from the DISC compared to vector alone transfected cells.

FIG. 6. Fas-inhibitor protein identified in lymphoma cells after immunoprecipitation of complex and LC/MS/MS analysis. A) Lymphoma cells were propagated in the presence of agonistic anti-Fas antibody to select for resistant cells and then to immunoprecipitate activated Fas. The remaining supernatant was precipitated a second time using an antibody that binds all Fas. The precipitants were analyzed by silver-stained gel, which showed the selective presence of a nonactivated Fas-associated band. B) The band was isolated and digested with trypsin, and the protein was identified by mass spectrometry. The identified peptide sequence aligns with the sequence of a p100 phosphoprotein.

FIG. 7. CD74 binds Fas and protects liver cells against apoptosis and animals against the lethal effects of anti-Fas antibody. A. Mice were transiently transfected with vectors to express CD74 (full length) FL, CD74 1-60 (truncated CD74) and immunoprecipitation/immunoblotting analysis were performed on liver extracts using anti-myc and anti-Fas antibodies. B. Mice were transiently transfected with vectors to express CD74 (full length) FL, CD74 1-60 (truncated CD74) and challenged with agonistic anti-Fas (Jo2). CD74 FL protect mice against the lethal effects of agonistic anti-Fas (Jo2) antibody. C. Shown are representative gross liver images of mice transfected with the constructs indicated and challenged with anti-Fas antibody. The vector-transfected CD74 1-60 transfected livers showed all or most tissues involved with hemorrhage and necrosis when examined under the microscope. The CD74 transfected liver shows a mostly normal pink appearance of liver with very few occasional sites of hemorrhaging and necrosis under the microscopic examination.

FIG. 8. Expression of CD74 deletion mutants shows binding to Fas in immunoprecipitation/immunoblotting analysis. (A) 293 cells were transfected with CD74 FL-myc and CD74 deletion mutants as indicated. (B) 18 hours after transfection, cell extracts were made and immunoprecipitation performed with anti-Fas antibody (B-10) and immunoblotting performed with anti-myc (9-E10 antibody). A suspected binding site of CD74 60-100 is suggested by this analysis.

FIG. 9. The amino acid sequence of CD74 and peptides with 5 amino acid overlap that represent a potential binding domain in CD74 that can be used to bind to Fas. Peptide LPKPPKPVSKMRMATPLLMQALPM blocks the mhc peptide binding domain. This may be because CD74 is expressed in dendritic cells apcs. This mechanism of blocking the peptide binding site of the MHC may be linked to cell survival. The same mechanism that provides survival signals to the cell via Fas may link to its ability to block MHC peptide binding.

FIG. 10. MSCV-GFP or MSCV-tdTomato lentiviruses can be used to express desired genes in BJAB (lymphoma) and KSSLK (Kaposi's sarcoma) cells and other cells. Introduction of virus allows high fraction infection of cells with the desired gene of interest.

FIG. 11. Cells were transfected with pHA-Fas FL-YFP (left) and pFlag-K1-mCer (right) and visualized by confocal microscopy (×100) to determine subcellular localization by merged images.

FIG. 12. Plasmids with genes coding for repeated peptides in tandem copies of DNA representing the Fas or p100 peptide-binding domain. We will track peptide by immunoblotting staining analysis with anti-HA and anti-GFP antibodies.

FIG. 13. Knockdown of CD74 in U-87 glioma cells makes them susceptible to the killing effects of temezolamide chemotherapy. The extent of CD74 suppression in stable CD74 shRNA levtivirus transfected cells (top). Cells with CD74 knockdown were more sensitive to the killing effects of chemotherapy (bottom).⁵³

FIG. 14. Expression of CD74 is common to several cancer types and is undetectable in peripheral blood lymphocytes. A-C) Levels of CD74 mRNA (A) and protein (B and C) are low in normal peripheral blood lymphocytes, but increase in early and late chronic lymphocytic leukemia (CLL) cells. D) Number of CD74-positive tumors by protein staining methods.⁴⁻⁸ E) The CD74 protein is absent in peripheral blood mononuclear cells of 10 donors and in Jurkat cells while BJAB cells express detectable levels of CD74.

FIG. 15. CD74 associates with inactive Fas and prevents binding of activating antibody CH-11 to Fas. Primary lymphoma cells were pre-incubated with CH-11 antibody and CH-11-associated Fas complexes were precipitated with anti-CH-11 antibody. Remaining supernatant was precipitated second time with anti-Fas antibody B-10 or protein A beads, and anti-myc as controls. CD74 associated exclusively with inactive Fas (B-10 lane) not active Fas (CH-11 lane) in primary lymphoma cells (top). Primary CLL cells were pre-incubated with CH-11 antibody and cell bound (Fas associated) CH-11 was detected by immunoblot. Remaining supernatant was precipitated with anti-Fas antibody B-10 to evaluate amount of Fas that failed to associate with CH-11(bottom).

FIG. 16. Peptides representing the membrane proximal region of CD74 facilitate Fas-mediated apoptosis. A. Sequences of four peptides spanning the membrane-proximal region of CD74; B. IP/IB analysis of the effect of peptides on association of CD74 with Fas (N5113—unrelated peptide control); C. Effect of CD74-derived peptides on Fc:FasL-induced apoptosis.

FIG. 17. A. Location on CD74 of 20mer peptides numbers 1 and 2, 9mer peptides numbers 1-4, and 7mer peptides numbers 1-3; B. Peptides 20mer 1 and 20mer 2 were effective in displacing the CD74 Fas complex in immunoprecipitation/immunoblot analysis; C. Seven-amino-acid length peptides (7mer 1, 7mer 2, and 7mer3) incubated with BJAB cells significantly enhanced levels of Fas ligand-induced apoptosis (earlier studies were done with agonistic CH-11 antibody) compared to buffer- and unrelated peptide-treated cells.

### DESCRIPTION OF ILLUSTRATIVE EMBODIMENTS

In one embodiment, the present invention relates to a method of treating a cancer characterized by inhibition of Fas-mediated apoptosis, comprising administering to a patient suffering from the cancer a pharmaceutically-effective amount of a composition comprising a peptide that competitively antagonizes at least one protein-Fas binding selected from the group consisting of herpes virus 8 protein K1 binding to Fas, CD74 binding to Fas, and p100 binding to Fas.

In one embodiment, the peptide is selected from the group consisting of peptides having from about 5 to about 35 amino acid residues, such as about 25 amino acid residues, and comprising at least a portion of the immunoglobulin domain of K1, peptides having from about 5 to about 35 amino acid residues, such as from about 5 to about 25 amino acid residues, and comprising at least five consecutive amino acid residues among positions 50-120 of CD74, LVTLLLAGQATTAYFLYQQQ (SEQ ID NO:2), LYQQQGRLDKLTVTSQNLQL (SEQ ID NO:3), QNLQLENLRMKLPKPPKPVS (SEQ ID NO:4), and PKPVSKMRMATPLLMQALPM (SEQ ID NO:5).

The sequence of CD74 is MHRRRSRSCREDQKPVMDDQRDLISNNEQLPMLGRRPGAPESKCSRGALYTGFSILV TLLLAGQATTAYFLYQQQGRLDKLTVTSQNLQLENLRMKLPKPPKPVSKMRMATPL LMQALPMGALPQGPMQNATKYGNMTEDHVMHLLQNADPLKVYPPLKGSFPENLR HLKNTMETIDWKVFESWMHHWLLFEMSRHSLEQKPTDAPPKESLELEDPSSGLGVT KQDLGPVPM (SEQ ID NO:1).

In one embodiment, the peptide has a sequence selected from the group consisting of LYQQQGRLD (SEQ ID NO:6), YQQQGRLDK (SEQ ID NO:7), QGRLDKLTV (SEQ ID NO:8), KLTVTSQNL (SEQ ID NO:9), QQQGRLD (SEQ ID NO:10), GRLDKLT (SEQ ID NO:11), and DKLTVTS (SEQ ID NO:12).

Generally, the peptide is unlikely to be the sole ingredient of the composition, though it may be. In one embodiment, the composition further comprises a pharmaceutically-acceptable carrier. By “pharmaceutically-acceptable” is meant that the carrier is suitable for use in medicaments intended for administration to a patient. Parameters which may considered to determine the pharmaceutical acceptability of a carrier can include, but are not limited to, the toxicity of the carrier, the interaction between the peptide and the carrier, the approval by a regulatory body of the carrier for use in medicaments, or two or more of the foregoing, among others.

The compositions can be made up in any conventional form known in the art of pharmaceutical compounding. Exemplary forms include, but are not limited to, a solid form for oral administration such as tablets, capsules, pills, powders, granules, and the like. In one embodiment, for oral dosage, the composition is in the form of a tablet or a capsule of hard or soft gelatin, methylcellulose, or another suitable material easily dissolved in the digestive tract.

Typical preparations for intravenous administration would be sterile aqueous solutions including water/buffered solutions. Intravenous vehicles include fluid, nutrient and electrolyte replenishers. Preservatives and other additives may also be present.

In the administering step, the composition can be introduced into the patient by any appropriate technique. An appropriate technique can vary based on the patient, the location and stage of the cancer, and the components of the composition, among other parameters apparent to the skilled artisan having the benefit of the present disclosure. Administration can be systemic, that is, the composition is not directly delivered to a tissue, tissue type, or organ at which the cancer is present, or it can be localized, that is, the composition is directly delivered to a tissue, tissue type, or organ at which the cancer is present. The route of administration can be varied, depending on the composition and the cancer, among other parameters, as a matter of routine experimentation by the skilled artisan having the benefit of the present disclosure. Exemplary routes of administration include transdermal, subcutaneous, intravenous, intraarterial, intramuscular, intrathecal, intraperitoneal, oral, rectal, and nasal, among others. In one embodiment, the route of administration is oral or intravenous.

A pharmaceutically-effective amount of the composition is one that imparts a dosage sufficient to treat the cancer. In one embodiment, administering comprises a dosage from about 1 μg peptide/kg body weight per day to about 1 g peptide/kg body weight per day.

A regimen for treating cancer will typically involve multiple dosages. In one embodiment, the administering step is repeated once every two to three days for a period of from about three to about twelve weeks. Other treatment regimens are possible and can be routinely selected by the ordinary skilled artisan.

Any cancer characterized by inhibition of Fas-mediated apoptosis can be treated by the present method. “Treat” is used herein, when referring to cancer, to any procedure fatal, either directly or indirectly, to at least one cancer cell, such as by removing inhibition of Fas-mediated apoptosis and thereby allowing apoptosis of cancer cells to proceed. In one embodiment, the cancer is selected from the group consisting of bladder cancers, breast cancers, central nervous system cancers, colorectal cancers, endometrial cancers, leukemias, lung cancers, lymphomas, melanomas, ovarian cancers, pancreatic cancers, and prostate cancers.

The method can further comprise administration of an apoptosis inducer to the patient. In one embodiment, the apoptosis inducer is radiation (such as at about 5 Gy). In one embodiment, the apoptosis inducer is stausporine.

In another embodiment, the present invention relates to a composition comprising a peptide that competitively antagonizes at least one protein-Fas binding selected from the group consisting of herpes virus 8 protein K1 binding to Fas, CD74 binding to Fas, and p100 binding to Fas. Such peptides have been described above. The composition can further comprise a pharmaceutically-acceptable carrier, as described above.

In another embodiment, the present invention relates to a method of treating a disease characterized by unwanted apoptosis in a mammal, comprising transfecting a cell of the mammal with a nucleic acid molecule encoding CD74.

CD74 is described above. Nucleic acid molecules encoding CD74 are known in the art or can be synthesized by the person of ordinary skill in the art having the benefit of the amino acid sequence of CD74 given herein. As will be understood by the person of ordinary skill in the art, the nucleic acid molecule encoding CD74 can be readily linked with appropriate regulatory sequences, e.g., promoters and the like, and packaged into an appropriate vector. Both regulatory sequences and the vector can be chosen by the person of ordinary skill in the art as a routine matter considering the mammal, the cell type within the mammal for which transfection is desired, and the transfection technique desired. The vector can comprise other coding regions than that encoding CD74, such as coding regions encoding selection markers, as is well known in the art. Transfection techniques are well known in the art. The person of ordinary skill in the art will recognize that a transfection technique may have a low, even a very low, rate of transfection (e.g., one cell in one thousand, one cell in one million, or even fewer may be transfected), but that transfected cells can be readily identified, isolated, and further processed; in other words, known transfection techniques are a matter of routine experimentation.

Any disease characterized by unwanted apoptosis can be treated by this method. In one embodiment, the disease is selected from the group consisting of lupus, liver toxicity, and toxic epidermal necrolysis (TEN).

As will be understood by the person of ordinary skill in the art, any reference herein to a method of treatment of a disease in a human or animal body by use of a particular composition inherently teaches use of the particular composition in the manufacture of a medicament for treatment of the disease.

Overview of Fas

The Fas receptor (FasR) is the most intensely studied member of the death receptor family. Its aliases include CD95, Apo-1, and Tumor necrosis factor Receptor Superfamily, member 6 (TNFRSf6). The gene is situated on chromosome 10 in humans and 19 in mice.

Isoforms

Previous reports have identified as many as eight splice variants, which are translated into seven isoforms of the protein. Many of these isoforms are rare haplotypes that are usually associated with a state of disease. Apoptosis-inducing Fas receptor is dubbed isoform 1 and is a type 1 transmembrane protein.

Function

Fas forms the Death Inducing Signalling Complex (DISC) upon ligand binding.

Membrane-anchored Fas ligand trimer on the surface of an adjacent cell causes trimerization of Fas receptor. This event is also mimicked by binding of an agonistic Fas antibody, though some evidence suggests that the apoptotic signal induced by the antibody is unreliable in the study of Fas signaling. To this end, several clever ways of trimerizing the antibody for in vitro research have been employed.

Upon ensuing DD aggregation, the receptor complex is internalized via the cellular endosomal machinery. This allows the adaptor molecule FADD to bind the death domain of Fas through its own death domain.

FADD also contains a death effector domain (DED) near its amino terminus, which facilitates binding to the DED of FADD-like ICE (FLICE), more commonly referred to as caspase-8. FLICE can then self-activate through proteolytic cleavage into p10 and p18 subunits, two each of which form the active heterotetramer enzyme. Active caspase-8 is then released from the DISC into the cytosol, where it cleaves other effector caspases, eventually leading to DNA degradation, membrane blebbing, and other hallmarks of apoptosis.

Role in Apoptosis

Some reports have suggested that the extrinsic Fas pathway is sufficient to induce complete apoptosis in certain cell types through DISC assembly and subsequent caspase-8 activation. These cells are dubbed Type 1 cells and are characterized by the inability of anti-apoptotic members of the Bc1-2 family (namely Bc1-2 and Bc1-xL) to protect from Fas-mediated apoptosis. Characterized Type 1 cells include H9, CH1, SKW6.4 and SW480, all of which are lymphocyte lineages except the latter, which is a colon adenocarcinoma lineage. However, evidence for crosstalk between the extrinsic and intrinsic pathways exists in the Fas signal cascade. In most cell types, caspase-8 catalyzes the cleavage of the pro-apoptotic BH3-only protein Bid into its truncated form, tBid. BH-3 only members of the Bc1-2 family exclusively engage anti-apoptotic members of the family (Bc1-2, Bc1-xL), allowing Bak and Bax to translocate to the outer mitochondrial membrane, thus permeabilizing it and facilitating release of pro-apoptotic proteins such as cytochrome c and Smac/DIABLO, an antagonist of inhibitors of apoptosis proteins (IAPB).

Restoration of Death Receptor Apoptosis

Advances in cancer treatment have been hampered by a limited understanding of the mechanisms blocking death receptor (Fas/CD95/Apo-1, TRAIL DR4 & DR5)-mediated apoptosis. For the over 20 years we have known that many cancer cells are resistant to Fas-mediated apoptosis only a small fraction have a basis for resistance. TRAIL receptor signaling is also widely variable suggesting this receptor function is highly amenable to regulation. Our research focus on apoptosis regulation is based on our finding that the K1 protein of oncogenic herpesvirus 8 binds to Fas and suppresses apoptosis and blocks the mouse death induced by anti-Fas antibody. We identified CD74 as associated with unactivated Fas in apoptosis-resistant cells. In preliminary analysis p100 and CD74 block Fas-mediated apoptosis in cells and prolong the survival of mice after challenge with a lethal dose of anti-Fas antibody. We hypothesize that cellular candidate inhibitors of death receptors (CD74 and other identified proteins) play a key role in blocking death receptors that can be targeted to improve cancer therapy.

We do not advocate the use of anti-Fas methods for cancer cell killing: instead we seek methods to selectively sensitize cancer cells to death receptor-mediated apoptosis, as previously demonstrated with biological agents IFNα anti-CD₂₀ antibody. The description of CD74 as an inhibitor of Fas will lead to the identification of groups of potential regulators of apoptosis that would explain widespread resistance.
1. We will show whether CD74 suppresses apoptosis in hematopoietic cancer cells. Apoptosis show where CD74 blocks induced with various stimuli including Fas-mediated apoptosis, radiation and stausporine. We will analyze proximal signaling steps in Fas-mediated apoptosis to identify site of apoptosis blockage by performing death inducing signaling complex analysis and using Fas-dependent inhibitors.
2. We will confirm that CD74 and Fas form a complex and map the binding site in CD74 used to bind Fas by expressing deletion mutants and expressing the minimal binding domain. The mapping of CD74 will be performed in mice by transfecting with deletion mutants of CD74 and demonstrating CD74 mutant-Fas complexes in cells and mouse liver tissue.
3. We will demonstrate whether peptides and protein domains representing the binding domain of CD74 can disrupt the CD74-Fas complex and whether disruption restores Fas signaling and apoptosis. Test the peptides in mice application to attempt disrupting the CD74-Fas complex to restore Fas signaling and apoptosis.

We will map the binding domain on CD74 and select the peptides that are most active in breaking up this complex. Furthermore, the establishment of apoptosis will be confirmed in a cell model and a mice model expressing the CD74-Fas complex. We will demonstrate a detailed understanding of death receptor and apoptosis regulation that will elucidate targets to facilitate apoptosis and enhance the effects of cancer chemotherapy. We have identified 3 potential death receptor inhibitors with activity and we will lay the foundation for apoptosis regulation of Fas and offer a novel therapy for cancer.

### BACKGROUND

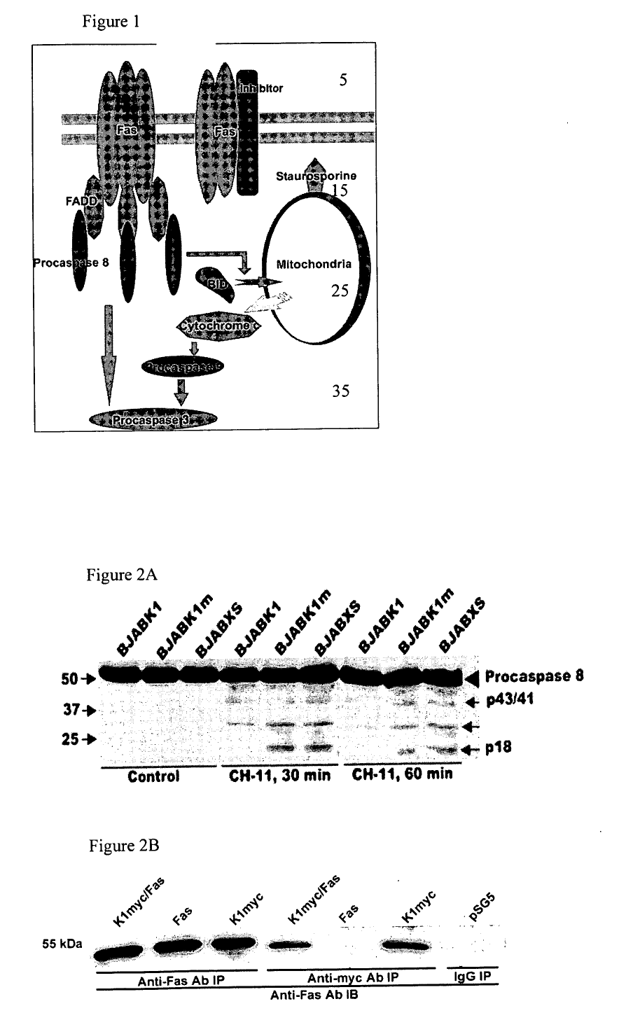

Fas resistance is a common and reversible problem in cancer cells. Cancer therapy is hampered by the prevalence of blockage of apoptosis. Normal and cancer cells have several apoptosis pathways that can be induced to selectively trigger killing of cancer cells. The Fas and TRAIL death receptor pathways are critical steps for life and death decisions of cells. These death receptor pathways can play key effector roles in mediating apoptosis of the intrinsic pathway (DNA damage, stress and chemotherapy). Thus, a fuller understanding of apoptosis should enhance our ability to induce apoptosis in cancer therapy. Apoptosis pathways are receptor mediated (Type I) or mitochondrial-mediated Type #). Resistance to receptor-mediated apoptosis can occur because of elevated cFLIP (e.g., Burkett's lymphoma), or expression of mutant Fas/FasL (e.g., NK/T-cell lymphoma). Sometimes resistance to Fas-mediated apoptosis is caused by low Fas expression levels, and the induction of high expression levels of Fas with interferon gamma (IFNγ) CD40L, or rituximab can restore Fas signaling and apoptosis. {de Totero, 2004 #2347; de Totero, 2003 #2352; Roue, 2001 #2317; Jones, 2001 #2316} These causes of blockage of Fas-mediated apoptosis explains only a small portion of resistance in cancer cells. For instance, hematologic cells have adequate expression of Fas, yet a few cells are sensitive to Fas-mediated apoptosis (FIG. 1).

We are not advocating through receptor activation apoptosis induction as a way to treat cancer because nonmalignant cells are sensitive to anti-Fas apoptosis; rather, we will selectively restore death receptor signaling in tumor cells by characterizing associated inhibitors. The presence of death receptor inhibitors may explain the widespread expression of death receptors and resistance to apoptosis in cancer.

Our showing that human herpesvirus 8 K1 binds and blocks Fas raises the question of whether tumor cells routinely expressed cellular proteins that bind and block death receptors. The properties of viral proteins (for example, vFLIP viral chemokine receptors) have guided investigators to the existence of cellular proteins that serve as targets of viral proteins and point to sites of critical cellular regulation. Though not to be bound by theory, because viral proteins evolve to mimic critical regulatory proteins, the Fas-inhibitor model is believed to represent how Fas is regulated by cellular proteins, too. We discovered that K1 binds and disables Fas (FIG. 1). In the proposed project we will search for and identify inhibitors of death receptors in cancer cells. Based on this model we have identified two cellular proteins, CD74 and CD74 that bind and block Fas-mediated apoptosis.

Preliminary Studies and Results

K1 suppresses anti-Fas antibody—mediated apoptosis, and we anticipate that K1 represents a family of transmembrane proteins that bind and regulate Fas. Retroviral transfected BJAB lymphoma cells treated with the agonistic antibody CH-11 (0-50 ng/mL). Show that K1 transfectants (BJABK1) had lower rates of apoptosis after 24 hours than K1m transfectants (BJABK1m) or vector-only transfectants (BJABXS). {Wang, 2007 #2374} K1 also protected THP-1 and U937 cells after stimulation with agonistic anti-Fas antibody but not when apoptosis was induced with radiation or tumor necrosis factor-α-related apoptosis-inducing ligand (TRAIL). {Okada, 1997 #1313; Pati, 2002 #1935}

Immunoprecipitation/immunoblotting analysis of the death-inducing signaling complex (DISC) showed lower levels of FADD and caspase 8 levels in K1-expressing cells (FIG. 2A), suggesting that K1 prevents DISC formation {Wang, 2007 #2374} while expressing constant levels of membrane Fas. We induced co-expression of K1 and Fas and performed immunoprecipitation/immunoblotting analysis with anti-Fas and anti-tag antibodies. We found that K1 complexed with Fas, including endogenous Fas (FIG. 2B). {Scaffidi, 1999 #2160; Song, 2005 #2076} We concluded that K1 binds to Fas and prevents Fas from being activated. Through expression of deletion mutants of K1 and addition of peptides representing the binding domain to cells, we mapped the binding site to the immunoglobulin domain present in the ectodomain of K1.

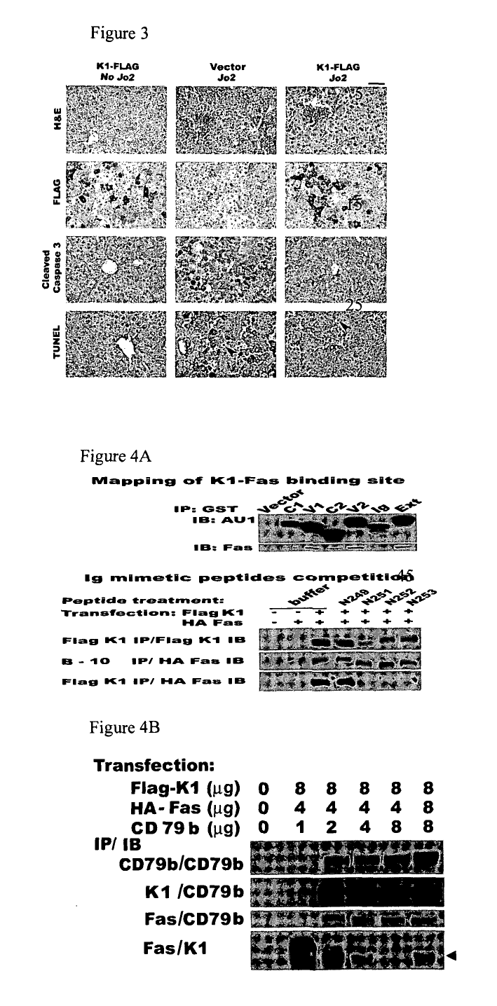

The K1-Fas regulation system operates in mouse tissues and protects mice from Fas-induced apoptosis. This model will be used to show how CD74 blocks apoptosis. To test whether K1 protects mice against the cell-killing mediated by Fas, we induced apoptosis by inoculating mice with agonistic anti-Fas antibody that kills mice by inducing widespread karyocyte apoptosis. K1-transfected mice and K1-transgenic mice had significantly higher survival rates than nontransgenic mice (FIG. 3). Jo2 antibody causes death of mice through induction of liver cell apoptosis. {Haga, 2003 #2098} We transfected mice with plasmids to express K1 and examined the liver tissue 6 hours after a lethal injection of Jo2 antibody. Transfected-K1 mice showed lower rates of hemorrhage and edema in the liver than did vector-transfected mice challenged with Jo2 antibody (FIG. 3). K1 mouse tissue shows lower rate of caspase 8 activity and Tuner straining cells. Immunoprecipitation/immunoblotting analysis of liver tissue extracts, showed that the K1 complexed with Fas (data not shown); this mouse model system allows evaluation in vivo of candidate inhibitors of apoptosis. We mapped the K1 binding domain used to bind Fas as being the immunoglobulin domain located in the ectodomain of K1 (FIG. 4). We overexpressed CD79A (an immunoglobulin-containing domain protein), which showed that this protein competed with K1-Fas binding (FIG. 4). We also applied synthetic 25mer peptides spanning the immunoglobulin domain of K1 and some of these peptides competed with the formation of the K1-Fas complex (FIG. 4). Furthermore, the competing peptides applied selectively facilitated K1 over control transfectants apoptosis induced by agonistic anti-Fas antibody (BJABK1 compared to BJABXS with N253 peptide was 77.3±4.5% and 46±4.5%) indicating this competing peptide enhance Fas mediated apoptosis whereas control peptide did not increase apoptosis in same cells with anti-Fas antibody (26±2 and 36±7.2%). This line of investigations indicate that inhibitors of death receptors can be identified, and that potential therapy with competing peptides representing the binding domain of inhibitors can include selective apoptosis of apoptosis resistant cells.

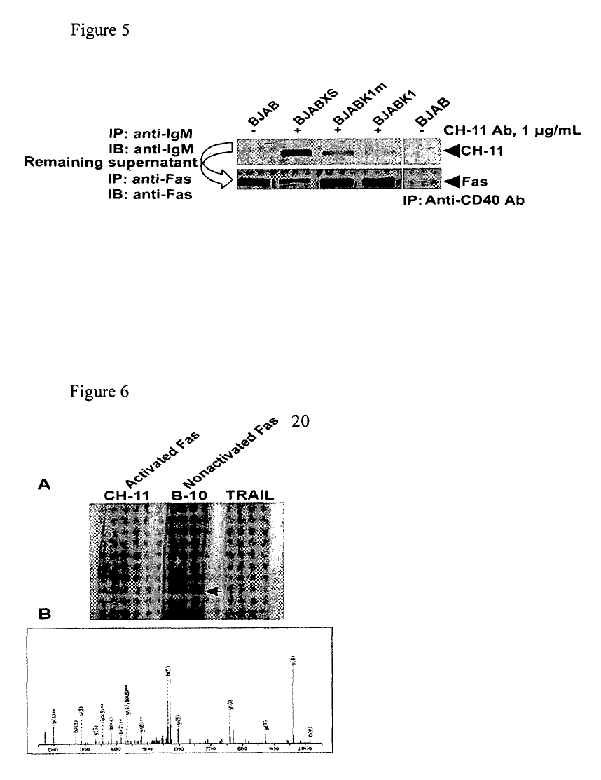

How does K1 block Fas mediated apoptosis? We suspect the K1 sequesters Fas and prevent Fas from participating in the death inducing signaling complex. Cells were activated with CH-11 antibody and the extracts were immunodepleted with the CH-11 antibody. The left over supernatant was precipitated and second time with anti-Fas antibody that binds to all Fas (inactivated Fas and Fas that was intracellularly at time of first CH-11 antibody incubation. We found that in K1 cells have less Fas is the DISC and most of Fas in away from the DISC compared to vector alone transfected cells. This indicates that K1 is sequestering Fas and preventing DISC formation (FIG. 5).

We identified inactive Fas associated proteins that are candidate inhibitors of Fas. There are substantial reasons for cells to express inhibitors of cell death receptors. {Wang, 2002 #1882} We searched for Fas inhibitors by first immmunodepleting cell extracts of activated Fas complexes (DISC) by activating receptor and precipitating with agnostic anti-Fas antibody CH-11. The remaining cell extract was precipitated a second time with the anti-Fas antibody B10, which reacts with all forms of Fas. We identified nonactivated Fas-associated bands that were absent in the antibody control lane (anti-TRAIL antibody) of the silver-stained gel (FIG. 6A). We have collaborated with Dr. R. Kobayashi at our institution to identify the Fas-binding protein using liquid chromatography and tandem mass spectrometry (LC/MS/MS) (FIG. 6B). As an example, the peptide identified from this bond is present in protein p100, which is highly expressed in tumors of different types and in growing blood vessels. We showed p100 blocked apoptosis induced by Fas antibody, binds to Fas, blocks apoptosis, protects against cell apoptosis and against death of mouse induced by anti-Fas antibody (data not shown). P100 is a target for regulating apoptosis through Fas and is also a target for anticancer therapy. A detailed description of p100 will therefore be withheld until further notice. We have identified several proteins associated with inactivated Fas in BJAB cells.

We have identified a third protein, CD74 as an inactive Fas-associated protein that is highly suspected to be a negative regulator of Fas in cancer cells. CD74 is the invariant subunit of the T cell receptor and is also expressed in numerous tumor cell lines and tumors.¹² CD74 functions as a MHC class II chaperone and accessory-signaling molecule, that facilitates endoplasmic reticulum exit of proteins. {Matza, 2002 #2424; Matza, 2003 #2423; Matza, 2001 #2425} Macrophage migration-inhibitory factor (MIF) activates CD74 and induces phosphorylation of the extracellular signal regulated kinase-½, cell proliferation, prostaglandin E2 production and nuclear transcription initiation. {Leng, 2003 #2419} We challenged 293 cells with anti-Fas (CH-11) antibody and found siRNA CD74-treated cells had significantly higher levels of apoptosis compared to control siRNA-treated cells (data not shown).

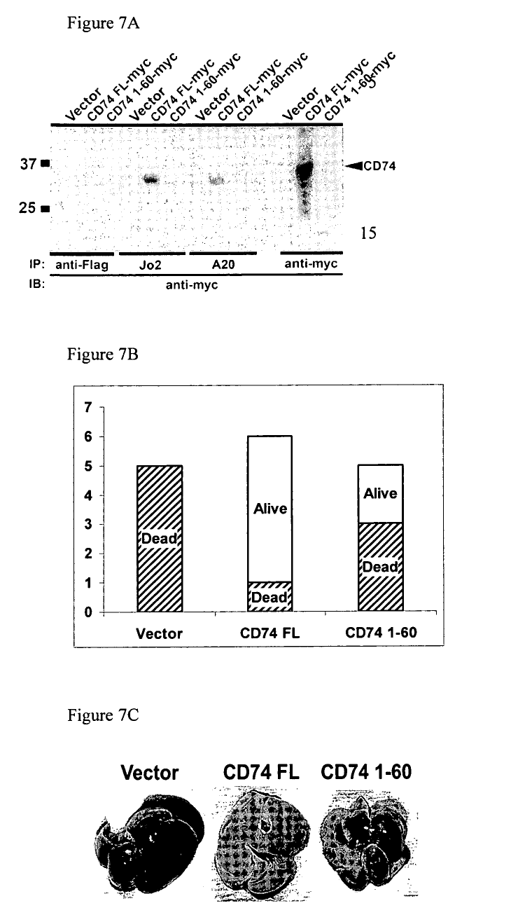

We transfected CD74 plasmid and control plasmid into mice and made extracts of liver tissue (tissue that highly expresses transfected genes and is amenable to regulation of Fas. Immunoprecipitation/immunoblotting analysis with anti-Fas and anti-CD74 antibody were performed on extracts which showed the association of CD74 with Fas (FIG. 7A). To determine whether CD74 blocked Fas, we transfected into mice with CD74 plasmid, CD74 1-60 (truncated), and vector alone. and challenged the mice with a lethal dose of Jo2 antibody. The CD74 mice had a significantly higher survival over CD74 1-60 and vector transfected mice (FIG. 7B). CD74 transfected liver had pink color normal liver tissue appearance, whereas-vector and CD74 1-60-transfected liver showed red-black color blotches (FIG. 7C) that showed hemorrhaging and necrotic tissue under microscope examination. Taken together these studies indicate that CD74 binds Fas in human and mouse cells and confers resistance to Fas-mediated apoptosis and mouse death. CD74 can be a potent inhibitor of Fas.

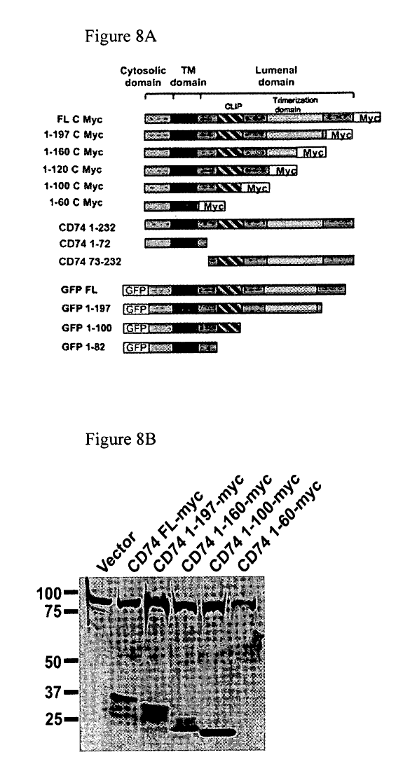

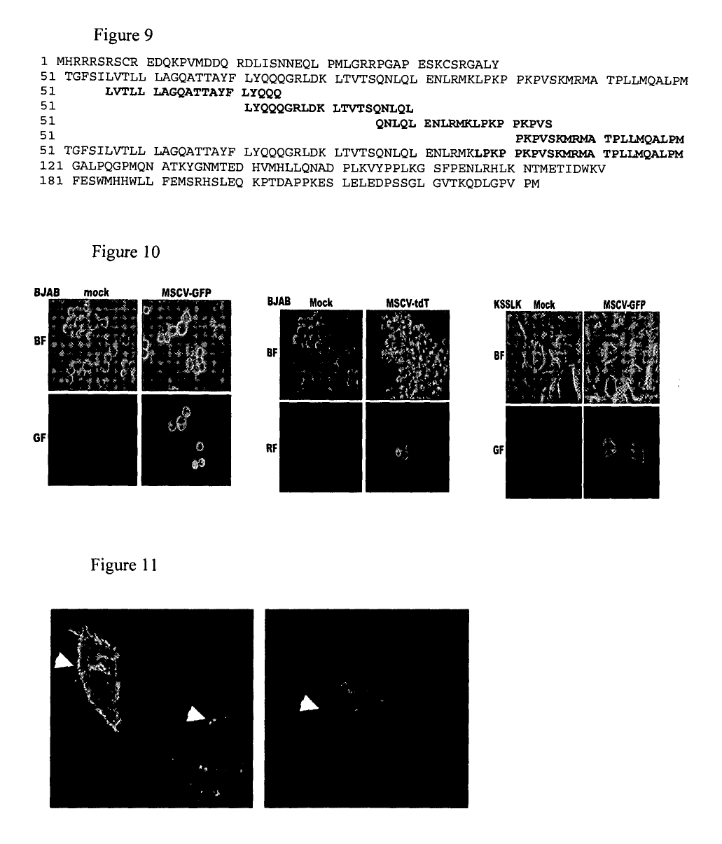

Using deletion mutants of CD74 we showed that the shortest construct does not bind Fas and amino acid 60-80 may be essential for binding Fas (FIG. 8). As with K1, we will use peptides representing this potential binding domain and determine if these peptides block CD74-Fas complex formation and induce apoptosis of cancer cells. Though not to be bound by theory, given the high expression of CD74 in human tumors (FIG. 9), we expect this approach will allow us to selective kill tumor cells by releasing CD74 inhibitor effect on Fas and induce tumor cell apoptosis.

CD74 can be used to protected cells against unwanted apoptosis. Lupus, liver toxicity, and toxic epidermal necrolysis (TEN) are medical conditions whose pathophysiology is mediated by activation of the Fas receptor. In these medical conditions, CD74 can be expressed in the desired tissues to inhibit apoptosis of cells. We have provided experimental data on mice transfected to express CD74 and the mice survive a lethal injection of agonistic anti-Fas antibody. Expression of CD74 renders cells resistant to the killing effects mediated by Fas. Therefore, diseases such as lupus, liver toxicity, and toxic epidermal necrolysis (TEN) can be treated by transfecting the cells with CD74 which will block Fas and maintain cell survival.

Treatment of patients with CLL with adenovirus expressing CD145 show enhanced levels of Fas but no immediate apoptosis.^{1,2} Given our background on CD74 regulation, we tested whether CD74 was upregulated with CD145. Stimulation with CD145 enhanced the levels of CD74 and, though not to be bound by theory, this may be why CLL cells do not die despite the enhanced production of Fas. This means that especially in CD145-treated cells, the use of the blocker of CD74 will have greatest effect in releasing the inhibitor CD74 and restoring apoptosis.

Knock down of CD74 protein restored Fas-mediated apoptosis of cells. Kitange et al recently reported that CD74 is highly expressed in chemotherapy-resistant gliomas and suppression of CD74 in glioma-derived U-87 cells by shRNAs using lentivirus system enhanced their response to chemotherapy (FIG. 13).³ This cell model may help in determining whether defective Fas signaling and apoptosis signaling can account for resistance to chemotherapy. To confirm whether CD74 interactions with Fas will interfere with Fas-mediated apoptotic signaling in lymphomas, we suppressed expression of CD74 in BJAB cells by transient transfection of CD74 siRNA (Ambion, Applied Biosystems, Foster City, Calif., and MTR Scientific, Ijamsville, Md.). Apoptosis was induced by incubating cells with agonistic anti-Fas antibody CH-11 48 hours post transfection. Control siRNA-transfected cell cultures showed an apoptosis rate of 38±7.8% compared with 54±9.8% (P=0.045) for CD74 siRNA-transfected cell cultures 24 hours after treatment with CH-11. Even at the apparent low level of transfection efficiency (10%), the apoptotic rate in CD74-suppressed cells increased significantly. The lentivirus CD74 knock down and transiently transient transfection system both indicated the role of CD74 in Fas mediated apoptosis and suggest that chemotherapy resistance is, in part, mediated by CD74.

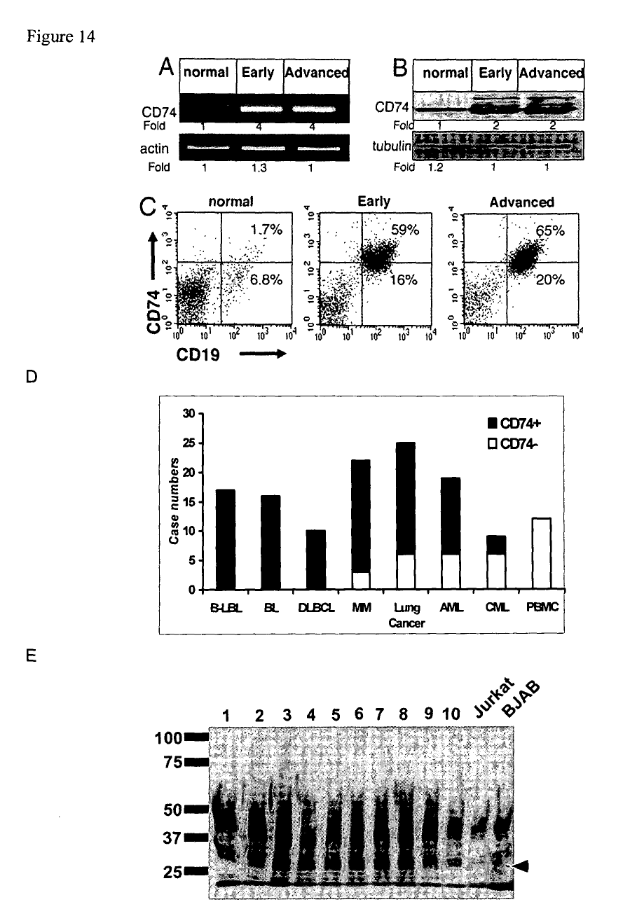

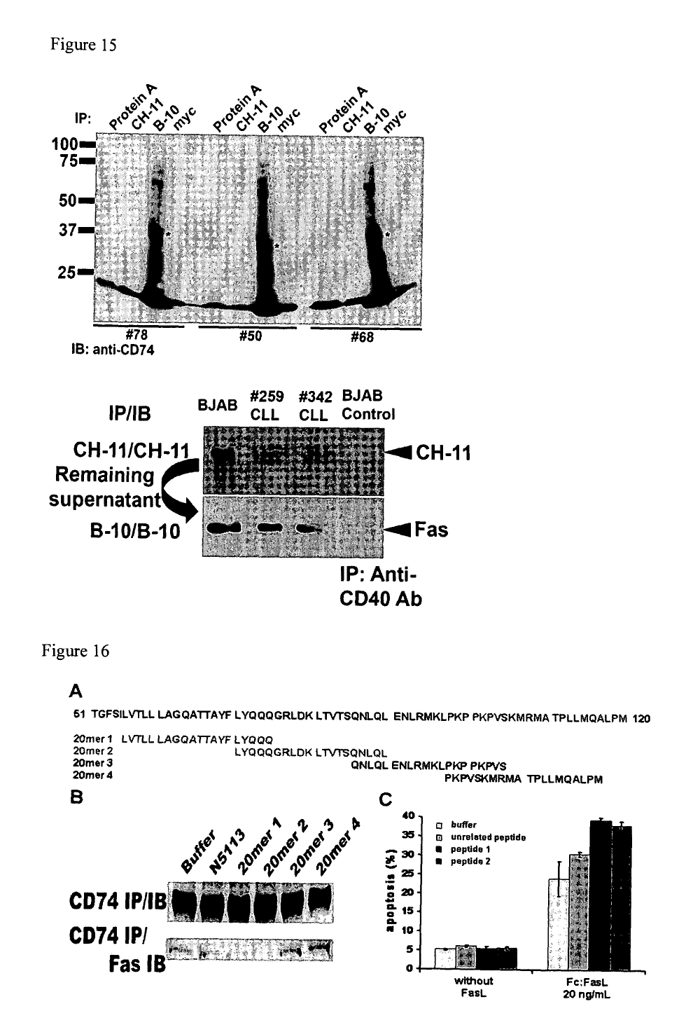

CD74 is expressed preferentially in hematopoietic cancers and is a key abnormality that can be targeted for therapy. CD74 is expressed preferentially in human lymphoma/leukemia tissues over non-tumor tissues (FIG. 14).⁴⁻⁸ Not only is CD74 associating with Fas in primary lymphoma cells, our results indicate that CD74 only associated with Fas that is not activated with CH-11 antibody, suggesting that CD74 binding of Fas has a primary role in Fas inhibition (FIG. 15). CD74 or another inhibitor may also explain the blockage of apoptosis in autoimmune disorders that express wild-type Fas.⁹ While autoimmune disorders have mutations in Fas and Fas ligand, a notable number of autoimmune disorders have wild-type Fas and Fas ligand expressed at normal levels and suggest a role for other agents including proteins that have a role in regulation of Fas signaling. Thus, there are reasons to anticipate a significant impact of CD74 on apoptosis regulation. We plan to use peptides representing potential Fas binding domain to block CD74-Fas complex formation and to restore apoptosis in hematopoietic cancer cells.

A membrane proximal region of CD74-derived peptides disrupts CD74-Fas complexes. Our preliminary data strongly suggest that the ectodomain of CD74 is required for association with Fas. Moreover, our data show that K1 and CD74 (FIG. 15; bottom panel) interfere with Fas activation by agonistic antibody CH-11 is due to interference with binding of CH-11 to Fas. The CH-11 binding site was mapped to the membrane-proximal CRD3-domain of Fas.¹⁰ We thus predicted that the membrane-proximal region of CD74 may be involved in the interaction with Fas. We designed four 20 amino acids long peptides with five amino acid overlaps spanning this region of CD74 (FIG. 16A). Two of the overlapping peptides were able to disrupt CD74-Fas complex (FIG. 16B) and also showed promising increase in apoptotic rate in response to Fas activation by Fc:FasL (FIG. 16C),¹¹ Whereas this enhanced apoptosis activity was observed with 100 uM concentration of peptides, we expect that shorter versions of this peptide will be able to accomplish equivalent results at several fold lower peptide concentrations.

Summary and relevance of the CD74 project. Our investigations have identified CD74 as a potential inhibitor of Fas that blocks Fas-mediated apoptosis in several systems. CD74-Fas interaction can be targeted by competing peptides as shown here or peptidomimetic agents to disrupt the CD74-Fas inhibitory complex and free Fas to restore cell apoptosis. This represents a potential treatment breakthrough for a large number of cancers of various histiologic types that express CD74 and show resistance to chemotherapy. The recent data presented by another lab suggests the chemotherapy resistance is mediated in part by CD74, independently substantiates the importance of CD74 in apoptosis regulation.

### BIBLIOGRAPHY & REFERENCES CITED

1. Chu P, Deforce D, Pedersen IM, et al. Latent sensitivity to Fas-mediated apoptosis after CD40 ligation may explain activity of CD154 gene therapy in chronic lymphocytic leukemia. Proc Natl Acad Sci USA. 2002; 99:3854-3859.
2. Kater A P, Dicker F, Mangiola M, et al. Inhibitors of XIAP sensitize CD40-activated chronic lymphocytic leukemia cells to CD95-mediated apoptosis. Blood. 2005; 106:1742-1748.
3. Kitange G, Carlson B, Schroede R M, et al. CD74 is overexpressed in cells from temozolamide resistant glioblastoma xenografts and the expression in patient samples is associated with poor overall survival. American Association of Cancer Research Annual Meeting April 12-16, San Diego. 2008:A3209.
4. Miles R R, Cairo M S, Satwani P, et al. Immunophenotypic identification of possible therapeutic targets in paediatric non-Hodgkin lymphomas: a children's oncology group report. Br J Haematol. 2007; 138:506-512.
5. Ioachim H L, Pambuccian S E, Hekimgil M, Giancotti F R, Dorsett B H. Lymphoid monoclonal antibodies reactive with lung tumors. Diagnostic applications. Am J Surg Pathol. 1996; 20:64-71.
6. Burton J D, Ely S, Reddy P K, et al. CD74 is expressed by multiple myeloma and is a promising target for therapy. Clin Cancer Res. 2004; 10:6606-6611.
7. Kaddu S, Zenahlik P, Beham-Schmid C, Kerl H, Cerroni L. Specific cutaneous infiltrates in patients with myelogenous leukemia: a clinicopathologic study of 26 patients with assessment of diagnostic criteria. J Am Acad Dermatol. 1999; 40:966-978.
8. Mandal S, Curtis L, Pind M, Murphy L C, Watson P H. S100A7 (psoriasin) influences immune response genes in human breast cancer. Exp Cell Res. 2007.
9. Dianzani U, Bragardo M, DiFranco D, et al. Deficiency of the Fas apoptosis pathway without Fas gene mutations in pediatric patients with autoimmunity/lymphoproliferation. Blood. 1997; 89:2871-2879.
10. Fadeel B, Lindberg J, Achour A, Chiodi F. A three-dimensional model of the Fas/APO-1 molecule: cross-reactivity of anti-Fas antibodies explained by structural mimicry of antigenic sites. Int Immunol. 1998; 10:131-140.
11. Schneider P, Bodmer J L, Holler N, et al. Characterization of Fas (Apo-1, CD95)-Fas ligand interaction. J Biol Chem. 1997; 272:18827-18833.

Relevance and Significance of Project

Advances in cancer treatment have been hampered by a limited understanding of the mechanisms blocking receptor-mediated-Fas (CD95/Apo-1 and TRAIL receptor)—mediated apoptosis of tumor cells. For over 20 years, we have known that hematopoietic cancer cells are resistant for the most part to Fas-mediated apoptosis despite the presence of Fas. These investigations will lead to identification of peptides and peptidomimetic agents that can release inhibitors of death receptors and reestablish cancer cell apoptosis to improve cancer therapy.

Methods

Our overall hypothesis is that cellular inhibitors of Fas have key roles in blocking Fas signaling and Fas-mediated apoptosis in SLL/CLL.

We will show whether CD74 suppresses apoptosis in hematopoietic cancer cells. Apoptosis will be induced with various stimuli including Fas-mediated apoptosis, radiation and stausporine. We will analyze proximal signaling steps in Fas-mediated apoptosis to identify site of apoptosis blockage by performing death inducing signaling complex analysis and using Fas-dependent inhibitors. We will use our experience with analyzing K1 to perform similar analyses of CD74 to test the working hypothesis that CD74 inhibits Fas-mediated apoptosis by interfering with DISC function. Our experimental approach will examine the formation of DISCs in the presence of CD74 expression and determine whether CD74 is associated with DISCs in immunoprecipitation/immunoblotting analysis. A series of different cell types and apoptosis inducers will be studied to determine the effect of CD74 effect on apoptosis. Upon completing the proposed studies, we expect to show that CD74 binds and sequesters Fas to prevent Fas signaling and apoptosis. Such findings will explain how Fas is blocked and how Fas resistance can be modulated to treat cancer.

CD74 and k1 expression and induction of apoptosis. For long-term expression of CD74 and K1 genes and their mutants we used in the retroviral vector pLXSN containing the K1 gene, CD74, and a reporter gene (alkaline phosphatase). We will use BJAB lymphoma cells (American Type Culture Collection, Manassas, Va.), U937, and THP-1 cells. Apoptosis of transfectants will be induced by incubation with 50 ng/mL anti-Fas antibody (CH-11). In case we have difficulties expressing any of the mutants in hematopoietic cell lines we have available in our lab two lentivirus systems (FIG. 10) which allow a fraction of cells to express the gene of interest.

We will show whether CD74 suppresses apoptosis induced by different stimuli such as receptor activation, cell stress, and DNA damage. We will stimulate apoptosis with anti-Fas antibody, FasL, TRAIL (extrinsic pathway), radiation, and staurosporine (mitochondrial or intrinsic pathway) to determine whether CD74 expression alters apoptosis. CD74-transfected BJAB cells in quadruplicate will be incubated with anti-Fas antibody (CH-11), Fas ligand, TRAIL (R&D Systems, Minneapolis, Minn.), or treated with 6 or 8 Gy of radiation. Radiation will be delivered using a Nasatron ¹³⁷Cs irradiator (US Nuclear, Burbank, Calif.). {Doostzadeh-Cizeron, 1999 #1829}

Expected Results and Alternative Approaches: With this approach, which we have used successfully in the past, we will determine whether CD74 suppresses apoptosis induced by different stimuli. After completing these studies, we will have determined whether CD74 lowers the levels of DISC subunit recruitment. We will be able to determine whether CD74-mediated inhibition of apoptosis is Fas-dependent. We expect to have identified cell types in which CD74 suppresses apoptosis. We will have confirmed whether K1 suppresses apoptosis that is receptor-mediated or mitochondrial-mediated.

Analysis of Apoptosis: We will use several methods to assess induction of apoptosis in transfected BJAB cells. (1) Cells will be stained with Hoechst 33342 dye and be scored for apoptotic morphology, (2) Genomic DNA will be analyzed for fragmentation, and (3) Cells will be stained with FITC-labeled Annexin V and 25 μg/mL propidium iodide and analyzed by FACS. {Samaniego, 1999 #816} We will perform this analysis in quadruplicate and compare apoptosis rates of K1, CD74, and vector-only transfectants to confirm that K1 and CD74 alter apoptosis.

Expected Results and Alternative Approaches: CLL are more difficult to transfect. For CLL cells we anticipate that expression of CD74 in CLL will require a lentiviral construct (FIG. 8). An alternative method would be to use a molecular-transfer method in which desired genes are expressed in human embryonic lung fibroblasts and to coculture those cells with CLL cells, which is expected to result in over 90% of CLL cells expressing the transgene. {Biagi, 2005 #2361}

Characterize DISCs with expression of CD74. CD74 blocks Fas-mediated apoptosis and we will examine the DISCs. We will use immunoprecipitation/immunoblotting analysis to precipitate Fas and then blot with an antibody against DISC subunits Fas, FADD, cFLIP, procaspase 8, and CD74myc in vector alone and CD74 myc-BJAB transfectants. We will use transfected CLL cells if we identify a method to efficiently transfect them. We will also determine whether stable transfectants of U937 and THP-1 cells show CD74-related suppression of apoptosis. {Pati, 2002 #1935; Moses, 1999 #951; Arvanitakis, 1996 #705}

Detailed Methods: Retrovirally-transfected BJABCD74myc and BJABXS cells will be induced to undergo apoptosis with anti-Fas antibody (50 ng/mL, CH-11) for 10, 20, and 30 minutes. Extracts will be made and immunoprecipitated with anti-IgM antibody. In the immunoblotting step, we will use antibodies against Fas, FADD, cFLIP, procaspase 8, (1C12, Cell Signaling Tech., Beverly, Mass.); Fas (B-10), FADD protein (H-181), c-FLIP, (G-11), and HA epitope (all from Santa Cruz Biotechnology, Santa Cruz, Calif.) DAXX (M-112 Santa Cruz, Biotech); hepatocyte growth factor receptor (HGFR) (95309; R&D Systems), and procaspase 10. {Peter, 2003 #1912; Peter, 1999 #2161},{Lee, 2003 #1885}, {Gottlob, 1998 #2214} K1 and HGFR-specific antibodies are positive controls.

Expected Results and Alternative Approaches: If CD74myc inhibits Fas signaling at the level of the DISC, we expect to find corresponding lower levels of some of the DISC subunits. We anticipate that cells that express CD74 will have lower rates of DISC subunits recruited to the complex. Typically 5 Gy of radiation and stausporine (______ mm) induces cell death in 50 to 60% of cells. {Fritz, 2003 #1941; Mirzaie-Joniani, 2002 #1942} This analysis will show whether CD74 blocks type II apoptosis or mitochondrial-based apoptosis. Apoptosis will be monitored by staining cells with Annexin V and propidium iodide, and analysis will be performed by FACS.

Determine whether CD74 binds to inactive Fas or activated Fas. In our preliminary studies we showed that CD74 binds to Fas and prevents Fas-mediated apoptosis signaling. In this study what we determine whether CJH74 blocks by physically binding to Fas at the DISC or away from the DISC. What we will do is transfect CD74 or MT plasmid into BJB1 Cells and induce the DISC formation with anti-Fas antibody. The binding Fas will be precipitated and the left over supernatant will then be precipitated a second time with anti-Fas antibody that reacts with all Fas (B10 antibody). Thus any Fas that was not activated with CH11 antibody will be recognized by the B10 antibody which will correspond to the inactive fraction. We anticipate that these studies will then confirm the results as we have seen in FIG. 5.

We will show whether CD74 promotes cell survival in vivo and mouse survival from Fas-mediated cell death. We anticipate that CD74, like K1, will protect against apoptosis when expressed in mice {Wang #2374}.

Detailed Methods: Four to six hours after intravenous injection of the Jo2 antibody (0.3 μg/gram mouse weight) into CD74-transfected mice and vector alone-transfected C57/B6 mice (5 mice per treatment group), we will section and then analyze the most sensitive tissue liver by terminal deoxynucleotidyl transferase-mediated dUTP nick-end labeling (TUNEL) assay and cleaved caspase 3 to compare the number of apoptotic cells in tissues of CD74-transfected and vector-transfected mice. Liver tissues will be examined after 6 and 8 hours for edema, hemorrhage, and apoptosis. The macroscopic appearance (e.g., regions of hemorrhage or edema) will be also compared (FIG. 5). Another group of transfected mice will be monitored for survival for 5 days after anti-Fas antibody injection. We have published results from the analysis of tissue apoptosis and in vivo Fas-mediated effects. {Samaniego, 1999 #816} Expected Results and Alternative Approaches: We anticipate that CD74-expressing liver cells will be protected against apoptosis and mouse death, as we have shown for K1. Taken together, the activity of CD74 in human cells, mouse cells, and mice will confirm whether CD74 suppresses Fas-mediated apoptosis.

Summary. Upon completion of the proposed studies, we expect to have shown whether CD74 suppresses Fas-mediated apoptosis, binds Fas, binds at DISCs, and interacts with Fas in vivo to block apoptosis and mouse death. Such information is important because we will have established a novel pathway for apoptosis regulation and will have identified potential sites for regulation of Fas apoptosis to enhance chemotherapy of CLL.

We will confirm that CD74 and Fas form a complex and map the binding site in CD74 used to bind Fas. The mapping of CD74 will be performed in mice by transfecting with deletion mutants of CD74 and demonstrating CD74 mutant-Fas complexes in liver tissue.

Here we will test the working hypothesis that CD74 interferes directly with Fas-mediated apoptosis. The experimental approach will be to express CD74 and Fas deletion mutants and monitor binding in immunoprecipitation/immunoblotting analysis and to identify peptides representing the binding domains that can compete with CD74-Fas binding to promote apoptosis. Completion of these studies will enable us to identify the binding domains of Fas and K1 by using peptides that compete for binding and induction of apoptosis.

Use CD74 deletion mutants to identify the minimal necessary sequence for binding Fas. Our objective is to identify a minimal segment of CD74 that binds to Fas in suppressing apoptosis. Our working hypothesis is that the CD74-Fas complex deletion mutants will assist in the mapping the binding domain. We will make deletion mutants of CD74 in strategy as we have done for Fas. We will induce the expression of deletion mutants of HA-CD74 and test for binding to Fas in a pull-down assay. We will refer to a Fas complex because other members besides Fas may be required for CD74 binding. Positive controls will precipitate K1 and HGFR, which binds Fas. Negative control antibody is anti-VEGFR antibody. We have three sets of deletion mutants of CD74 some of which are conjugated with GFP (FIG. 8). This will be used to complement mapping with one set of deletion mutants compared to the results with one of the 3 sets of deletion mutants (FIG. 8).

Expected Results and Alternative Approaches: Mapping the CD74-Fas binding site will be additionally confirmed by anti-Fas or anti-CD74 blocking antibodies targeting the binding domain. If there are no commercial antibodies available and this becomes essential information for mapping and potential therapy, we will make antibodies under a different research project. We will compare results with peptide competition results to confirm results from experiments with CD74 deletion mutants and blocking antibodies.

Identify the Fas domain that interacts with CD74 to modulate apoptosis. Will not be attempted in this project. We have made Fas deletion mutants and all the Fas deletion mutants appear to interact in what appears to be a nonspecific manner with other proteins. We suspect that because the extracellular domain of Fas has many cysteines and production of deletion mutants will produce protein aggregation or force normative protein folding affecting binding sites at a distance and nonspecific manner. Others have reported this same difficulty when attempting to map on Fas binding domains. Thus for the moment we will hold on this approach until we find a better and simpler way to map binding sites on Fas without creating nonspecific binding properties. If we figure out a way to block nonspecific interaction of Fas deletion mutants we approach again the project on mapping on Fas the binding site. Some mapping studies that use deletion mutants are prone to difficulties because the altered protein conformation may affect interactions at distant sites. Subsequently, we may face a more difficult task with deletion mutants having distal effects on other regions of the protein because of disrupted protein binding. Therefore results from this approach will be corroborated with results from peptide competition analyses and a dual-staining immunohistochemical approach to confirm association.

We will identify CD74 peptides that represent the binding domain and disrupt the CD74-Fas association. These experiments are essential to the overall strategy of using therapeutic peptides to restore Fas signaling and apoptosis. Once the CD74-binding domain is identified, we will use peptides representing the binding domain to attempt blockage of the Fas-protein complex.

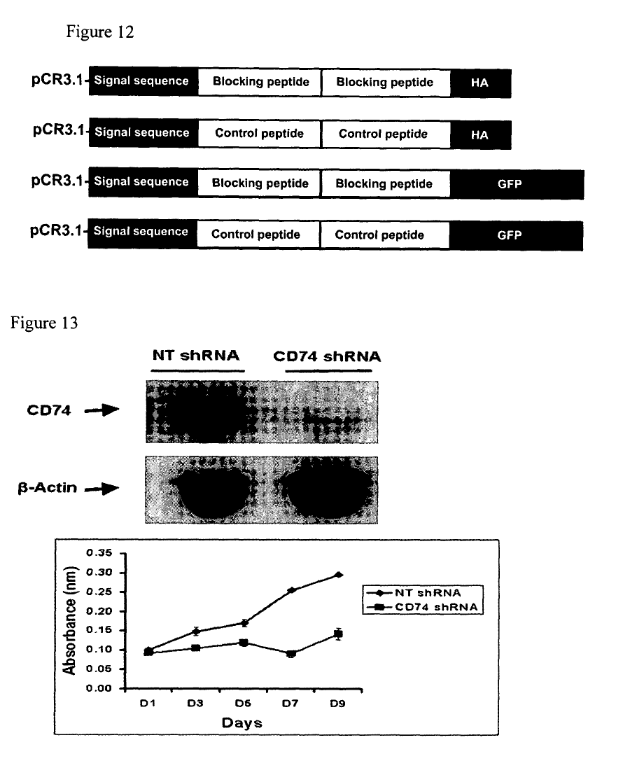

Detailed Methods: We will synthesize 25-mer peptides with 5-amino acid overlaps that span the CD74-binding domain of Fas. These peptides will have modified termini that protect against proteolytic digest (letter of collaboration, Dr. R. Arlinghaus, Ph.D. Anderson Peptide Core Facility). {Rehfeld, 1998 #2089; Dockray, 1987 #2090; Tanaka, 2003 #2091} We will incubate BJAB cells transfected with CD74myc and vector alone in RPMI and reduced FBS (0.1%) in the presence of 1, 5, and 10 μM peptides for 10 minutes. Control peptides will be mouse random sequence peptides of the same length. CD74myc-Fas complex formation will be monitored by immunoprecipitation/immunoblotting analysis. Once we identify a candidate peptide that disrupts the CD74myc-Fas interaction, we will confirm association by colocalization with monoclonal anti-HA (HA-CD74) and polyclonal anti-Fas antibodies (9E10, sc-40; B10, sc-715, Santa Cruz) or by expression of tandem copies of peptide fused with GFP (FIG. 12). Toxicity of peptides to BJAB cells will be measured by the levels of extracellular lactate dehydrogenase in the culture media using ELISA. {Nerurkar, 2005 #2198}

Expected Results and Alternative Approaches: Identifying competing peptides that are effective in cell systems has been largely unsuccessful because of the difficulty in delivering of peptides to targets inside cells. Our target is a protein complex formed through the extracellular domains of the proteins, which should facilitate accessibility of target. CD74 can be found on the surface of cells. Nonspecific binding sites for peptides will be overcome by preincubating cells with 10 μM scrambled sequence peptide. Peptides will be capped with nondegradable residues at the termini to resist to proteolysis. {Adessi, 2003 #2182} The Peptide Core Facility at M. D. Anderson routinely produces modified, degradation-resistant peptides dedicated to for this purpose. {Rehfeld, 1998 #2089; Dockray, 1987 #2090; Tanaka, 2003 #2091} The binding domain may consist of an area that represents a distant location in the linear protein sequence. If this is the case, short peptides of the protein sequence will not be effective competitors for CD74-Fas binding, and we will use these methods to determine the binding domain. To confirm results of peptides and to use another approach for delivery of peptides we will transfect cells with plasmids the express the peptide of interest in tandem (FIG. 12). This approach will express high local concentration of the peptide and also express peptide intracellularly where receptor-receptor interaction are often initiated in maturation of receptor complexes.

We express proteins with minimal binding domain amino acid 60-100 to show that expression of this domain can compete with CD74-Fas complex formation. Our preliminary studies (FIG. 8) show that the amino position 60-100 is essential for binding Fas. We have been able to confirm these studies through expression of various deletion mutants. In this section we will confirm these results by overexpressing the domains in cells and showing that you can compete with the expression of the domain and compete with the CD74-Fas complex. Expression of this domain will be performed in 293 cells with vector control. Eighteen hours after transfection the cell extracts will be immunoprecipitated for Fas and then blotted for CD74 using the myc tag. The analysis will be carried out in IP/IB for CD74 and Fas with increasing levels of transfected binding domain plasmid. Because the domain has an epitope tag we will stain cells to determine colocalization with CD74 and Fas.

Summary. When the proposed studies have been completed, we expect to have identified the binding domains on CD74 and Fas and peptides that compete with this interaction. In a subsequent project, we will have determined whether competing peptides block CD74-Fas interaction and induce apoptosis in cells. Such findings are important for designing plans for treating patients with Fas-resistant CLL.

We will use peptides representing the binding domain of CD74 to attempt competing with the CD74-Fas complex. Show that whether breaking the CD74-Fas complex also restores Fas signaling in hematopoietic cancer cells. We will determine whether peptides reconstitute apoptosis in a cell culture system. Cells that express wild-type CD74 and Fas will be incubated with effective concentrations of peptide as well as peptide control (scrambled or reverse sequence peptides) for 20 minutes at room temperature. Cells will be induced to undergo apoptosis with anti-Fas antibody and then after 20 minutes of incubation at room temperature cells will be washed and placed in growth medium. The cells will be monitored at 24, 36 and 48 hours for expression of CD74-Fas complexes by immunoprecipitation/immunoblotting analysis. The average we compared to 5 wells of control peptide cells. Toxicity will be analyzed by the addition of both of these peptides to cells without anti-Fas antibody. We do not foresee any problems with this type of analysis. We have performed reconstitution of Fas-mediated apoptosis in cells that express K1 and showing that we can break the complex and induce apoptosis. In the analysis of these results we will confirm that these results can be achieved also by peptide synthesis within cells using transfected plasmids directing the expression of copies of the competing peptide. These constucts will be produced in a stable expression system with selected cells made to express the peptide of interest. This system will bring in new challenges, for example that the synthesis peptide leads to delivery of the peptide to the right subcellular location. In this case it will be the endoplastic reticulum followed by targeting the extracellular domain of these complexes. We will be able to track the peptide production by placing an epitope tag to the peptide and using analysis in tracking the synthesis of the peptide using extracts for ELISA or Western Blotting system. At the conclusion of these studies we will have used two systems, one a cell expressed plasmid system as well as a synthetic peptide added to cell system to indicate whether indeed peptides complete with the CD74-Fas complex formation reconstitute Fas signaling in cells. The results from these studies will also be interpreted in the context of this peptide activity in in vivo studies as described herein. The peptides will be tested in vivo by application of peptides in liver tissue concurrently with induction of anti-Fas mediated apoptosis to determine whether breaking the CD74-Fas complex restores apoptosis in vivo.

We will incubate cells treated with siRNA for CD74 and control siRNA with peptides to attempt sensitization of cells to Fas mediated apoptosis. We have performed this experiment with peptides targeting K1 (FIG. 4). The cells will be incubated for 24-48 hours and apoptosis rates of treated cells will be analyzed. Correlative studies will be done in these cells to show whether peptide treatment breaks the CD74-Fas complex in IP/IB analysis.

Expected Results and Alternative Approaches: Both siRNA and peptides may by themselves be toxic to cells so controls with each will done to assess their relative contribution.

We will test whether peptides expressed or administer to mice with tumor will facilitate tumor eradication. CD74 expressing cells will be transduced with lentivirus expressing tandem copies of the active peptide and marker (FIG. 10) using lentivirus vector established in our lab. These peptide expressing tumorigenic cells will be implanted in SCID mice (10 mice per group) and once a 1 cm tumor is establish mice will be treated with subtherapeutic dose of anti-human Fas (CH-11) which detects human and not mouse Fas {Hiramoto, 2006 #2471}.

Expected Results and Alternative Approaches: If peptide expression from lentivirus facilitates Fas apoptosis as in our other experimental models, we should see tumor regression in peptide bearing tumor over control tumors.

Statistical considerations. Quantitative results generated by our studies will be in the form of cell counts and densitometer readings. We will calculate means and standard deviations. When the data are normally distributed, the Student t test will be applied to estimate the statistical significance of mean differences. {Daniel, 1974 #185} All P values will be determined using two-sided tests, and P<0.05 will be required to consider results as significant. We have found that the range of interexperimental variability in tumor induction and intervention experiments requires the use of at least 5 mice per treatment group. {Samaniego, 1995 #33} Mouse survival between treatment conditions will be compared with Wilcoxon (Gehan) statistical tests. {Wilcoxon, 1945. #2145}

After completing these studies, we will show how a newly identified inhibitor of Fas blocks apoptosis. We will know the binding domains and demonstrate how CD74 blocks apoptosis in mice. These conclusions will form the basis of a novel perspective on apoptosis regulation and also on the design of peptides that compete with CD74-Fas binding and show how peptides (or peptidomimetic agents) can break the CD74-Fas interaction present in CLL and other leukemia, thus sensitizing cells to Fas-mediated apoptosis and enhancing killing by chemotherapy. This research, in an area ignored by previous traditional chemotherapy approaches, is important because it takes advantage of new methods to turn on apoptosis selectively in cancer cells.

Method of Treatment with Antagonists of Fas Inhibitors

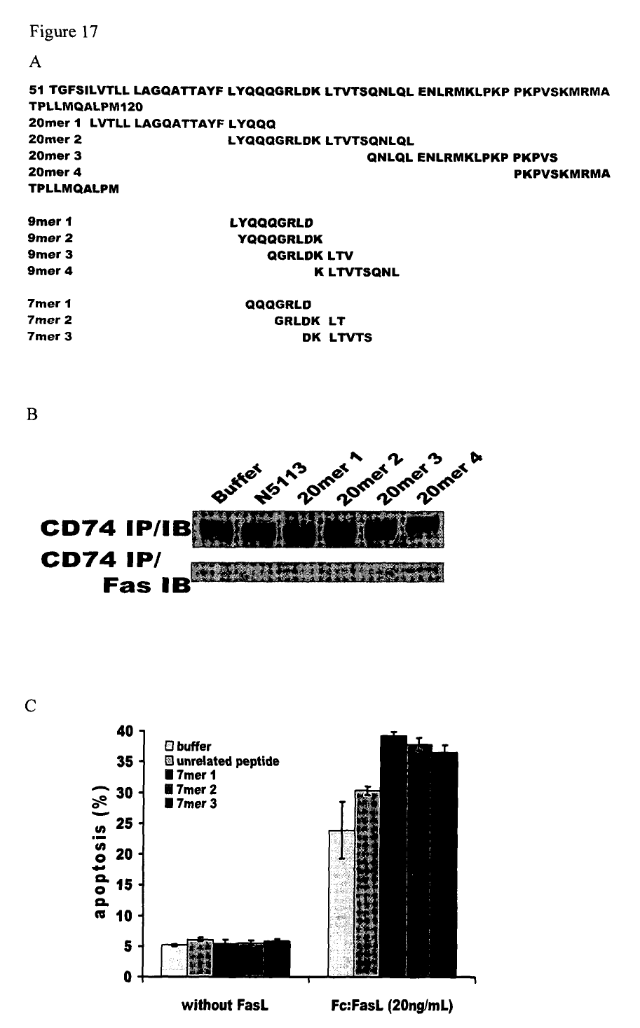

The goal of this example was to design and identify peptides that compete with the Fas-CD74 complex in order to enhance Fas-mediated apoptosis. FIG. 17A shows the location on CD74 of 20mer peptides numbers 1 and 2 that were shown above to displace CD74-Fas complexes. Peptides 20mer 1 and 20mer 2 are effective in displacing the CD74 Fas complex in immunoprecipitation/immunoblot analysis (FIG. 17B). The two peptides also enhanced Fas-mediated apoptosis. In order to better define competing peptides and peptides with apoptosis activity, we tested shorter peptides. We incubated seven amino acid length peptides (7mer 1, 7mer 2, and 7mer3) with BJAB cells and then stimulated apoptosis using recombinant Fas ligand (Fc:FasL). After 24 hours, the 7mer 1, 7mer 2 and 7mer 3 peptides showed significantly enhanced levels of Fas ligand-induced apoptosis (earlier studies were done with agonistic CH-11 antibody) compared to buffer- and unrelated peptide-treated cells (FIG. 17C).

Thus these results show short (7 amino acid) peptides representing the binding domain of CD74 enhanced Fas-mediated apoptosis. Fas-mediated apoptosis is a component of killing cells in chemotherapy treatment of cancer. These studies indicate that peptides may be used to modulate regulator proteins such as the Fas death receptor and enhance the killing effects of chemotherapy.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of treating a cancer characterized by inhibition of Fas-mediated apoptosis, comprising:
administering to a patient suffering from the cancer a pharmaceutically-effective amount of a composition comprising a peptide that competitively antagonizes at least one protein-Fas binding selected from the group consisting of herpes virus 8 protein K1 binding to Fas, CD74 binding to Fas, and p100 binding to Fas.

2. The method of claim 1, wherein the peptide is selected from the group consisting of peptides having from about 15 to about 35 amino acid residues and comprising at least a portion of the immunoglobulin domain of K1, peptides having from about 5 to about 35 amino acid residues and comprising at least five consecutive amino acid residues among positions 50-120 of CD74, LVTLLLAGQATTAYFLYQQQ, LYQQQGRLDKLTVTSQNLQL, QNLQLENLRMKLPKPPKPVS, and PKPVSKMRMATPLLMQALPM.

3. The method of claim 1, wherein the cancer is selected from the group consisting of bladder cancers, breast cancers, central nervous system cancers, colorectal cancers, endometrial cancers, leukemias, lung cancers, lymphomas, melanomas, ovarian cancers, pancreatic cancers, and prostate cancers.

4. The method of claim 1, wherein the composition further comprises a pharmaceutically-acceptable carrier.

5. The method of claim 1, wherein administering comprises a dosage from about 0.001 mg peptide/kg body weight per day to about 1 g peptide/kg body weight per day.

6. The method of claim 1, further comprising administration of an apoptosis inducer to the patient.

7. A composition, comprising:
a peptide that competitively antagonizes at least one protein-Fas binding selected from the group consisting of herpes virus 8 protein K1 binding to Fas, CD74 binding to Fas, and p100 binding to Fas.

8. The composition of claim 7, wherein the peptide is selected from the group consisting of peptides having from about 15 to about 35 amino acid residues and comprising at least a portion of the immunoglobulin domain of K1, peptides having from about 5 to about 35 amino acid residues and comprising at least five consecutive amino acid residues among positions 50-120 of CD74, LVTLLLAGQATTAYFLYQQQ, LYQQQGRLDKLTVTSQNLQL, QNLQLENLRMKLPKPPKPVS, and PKPVSKMRMATPLLMQALPM.

9. The composition of claim 7, further comprising a pharmaceutically-acceptable carrier.

10. A method of treating a disease characterized by unwanted apoptosis in a mammal, comprising:
transfecting a cell of the mammal with a nucleic acid molecule encoding CD74.

11. The method of claim 10, wherein the disease is selected from the group consisting of lupus, liver toxicity, and toxic epidermal necrolysis (TEN).

12. Use of a composition comprising a peptide that competitively antagonizes at least one protein-Fas binding selected from the group consisting of herpes virus 8 protein K1 binding to Fas, CD74 binding to Fas, and p100 binding to Fas in the manufacture of medicament for the treatment of a cancer characterized by inhibition of Fas-mediated apoptosis.

13. Use of a nucleic acid molecule encoding CD74 in the manufacture of a medicament for the treatment of a disease characterized by unwanted apoptosis.
